(19) United States
(12) Patent Application Publication
(10) Pub. No.: US 2026/0102091 A1
(43) Pub. Date: Apr. 16, 2026

(54) LANCING DEVICES

(71) Applicant: Marldell Limited, Petersfield (GB)

(72) Inventor: Steve KUESTER, Petersfield (GB)

(21) Appl. No.: 19/112,339

(22) PCT Filed: Sep. 25, 2023
(86) PCT No.: PCT/GB2023/052480

§ 371 Date: Mar. 17, 2025

(30) Foreign Application Priority Data
Sep. 26, 2022 (GB) 2214029.7

(51) Int. Cl. A61B 5/15; A61B 5/151

(52) U.S. Cl. CPC A61B 5/150412; A61B 5/150022; A61B 5/150549; A61B 5/150618; A61B 5/150717; A61B 5/150916; A61B 5/15117; A61B 5/15128; A61B 5/15144

(57) ABSTRACT

The invention relates to a single use lancing device comprising: a housing defining a cavity; a needle assembly comprising a needle and a needle holder, the needle assembly located within the cavity; a release member comprising: a body portion, the body portion having an aperture passing through the body portion along a central longitudinal axis of the release member from a rear opening to a forward opening; and retaining arms; a release member cap configured to be received by the rear opening such that the release member cap at least partially fits within the aperture; and a spring arranged to bias the needle assembly in a forward direction relative to the release member.

### FIELD OF THE INVENTION

The present invention relates to lancing devices, in particular single use lancing devices with a lancet that is retracted into a shielded position after use.

### BACKGROUND

Lancing devices are commonly used both in medical facilities and by private individuals to obtain a blood sample from a subject by puncturing the skin of the subject. Lancets are used when only a small amount of blood is required. Subjects that have diseases such as diabetes use blood testing at regular intervals to monitor blood sugar levels, and many other diseases and conditions require small samples of blood to be taken, for example for monitoring or diagnostic purposes. Obtaining a sample of blood usually involves pricking the skin on a finger or other suitable body part with a lancet or needle. In view of blood-borne diseases, it is important that, after use of the lancet, parts of the lancet that have been in contact with blood do not then come into contact with other persons. Thus, an important aspect of lancing devices involves preventing the lancet or needle from wounding another person after the skin of the patient has been punctured. The lancet should therefore be shielded, after use of the device, to prevent accidental wounding of another person. Further, the lancing device should be disposable to reduce the possibility of disease transmission due to the device being used subsequently on other persons. In this respect, the lancet should preferably be a single use device with features to prevent reuse.

Contact activated lancets are known in the art. These devices have a needle holder and a triggering device enclosing a lancet structure. They are maintained in an armed position ready to be activated by pressure on the triggering device. Drawbacks of known lancing devices include the problem of accidentally triggering the lancet, either because the lancing device is designed to be triggered on contact with the skin or because the triggering device is activated accidently by the user before the lancet is correctly positioned on the skin. Many single use lancets are wasted because they are activated before they are in the correct position and therefore do not pierce the skin adequately to obtain a suitable blood sample.

Several prior art lancing devices attempt to overcome the problem of accidental triggering by including complicated arrangements for retaining and releasing the lancet within the structure of the lancing device. These complex arrangements require the lancing device to be comprised of many components. Therefore, these lancets are often expensive and therefore not suitable for mass market use. These arrangements are also complicated to manufacture. Often the triggering device acts to force the lancet apart as it is being assembled. For example, spring activated triggering mechanisms may be difficult to retain in position during assembly.

It is therefore desirable to provide a robust, disposable lancing device of simple construction and assembly which effectively reduces the likelihood of the lancet triggering accidentally.

### SUMMARY

According to a first aspect of the invention, there is provided a single use lancing device. The lancing device comprises a housing defining a cavity; a needle assembly comprising a needle and a needle holder, the needle assembly located within the cavity; and a release member. The release member comprises: a body portion, the body portion having an aperture passing through the body portion along a central longitudinal axis of the release member from a rear opening to a forward opening; and retaining arms. The lancing device further comprises: a release member cap configured to be received by the rear opening such that the release member cap at least partially fits within the aperture; and a spring arranged to bias the needle assembly in a forward direction relative to the release member.

The release member cap may comprise a latching mechanism configured to engage with the release member such that when the release member cap is received by the rear opening, removal of the release member cap via the rear opening is prevented.

The release member and/or release member cap may further comprise a stopping mechanism configured to prevent the release member cap being advanced through the aperture beyond a desired position.

The latching mechanism and stopping mechanism may comprise a protrusion on an inner surface of the release member aperture and a corresponding recess on an outer surface of the release member cap. The release member protrusion may be received by the recess of the release member cap such that when the cap is positioned at a desired position within the release member, forward and backward movement of the cap relative to the release member is prevented. Alternatively, the cap may be provided with a protrusion that is received by a recess in the inner wall of the aperture.

The latching mechanism may comprise a projection or latch on the release member cap that is configured to engage with a portion of the release member. The projection or latch may be located on the cap such that when the cap is passed through the aperture to a desired position, the latch advances past the forward opening, thereby engaging with a front face of the release member body and preventing the cap being moved back through the aperture.

The stopping mechanism may comprise a tapered portion of the aperture, or a portion of the aperture with a smaller diameter than at the rear opening. The release member cap or a portion thereof may be sized so that it cannot be advanced through the aperture from a rearward direction beyond a desired position. Alternatively or additionally, the release member cap may be tapered; a front end of the cap having a smaller diameter than a rear end.

The release member cap may further comprise at least one guide rib and the aperture of the release member may comprise at least one corresponding guide rail, such that the guide rib slidably moves along the guide rail when the release member cap is received by the aperture. Alternatively, the cap may comprise a guide rail and the aperture may comprise a guide rib.

The release member cap may be configured to seal the aperture. The release member cap may be solid piece with an outer diameter that is substantially the same as the inner diameter of the aperture. Alternatively, the release member cap may not completely seal the aperture from the rear direction. The release member cap may be hollow or have an aperture passing through the length of the cap.

The aperture of the release member may be configured to allow the needle assembly and spring to be passed through the aperture from the rear opening to the front opening.

Providing the release member with an aperture and a cap may advantageously provide for easier assembly of the lancing device compared to devices without these features. The aperture of the release member body may allow the needle assembly and spring to be passed through the aperture to an assembled position wherein the needle assembly is positioned between the retaining arms. The aperture may act to align the needle assembly with the arms, necessarily ensuring that the device is assembled correctly. In a device without an aperture and cap, the needle assembly would need to be inserted into the retaining arms from the front or side of the release member. This may involve prising the retaining arms apart or having to manually align the retaining arms with surfaces of the needle assembly.

The lancing device of the present invention may be assembled in the housing. The release member may be inserted into the housing, and then the needle assembly and spring may be ‘dropped in’ via the aperture. The release member cap may then be inserted into the aperture to retain the components. This configuration may further simplify assembly of the device. The housing may further act to align the components or retain the needle assembly between the retaining arms. Such assembly would not be possible in a device wherein the needle assembly must be inserted into the release member from the front or side.

The needle assembly may comprise at least one guide rib and the aperture of the release member may comprise at least one corresponding guide rail, such that the needle assembly guide rib slidably moves along the guide rail when the needle assembly is passed through the aperture. Alternatively, the needle assembly may comprise a guide rail and the aperture may comprise a guide rib.

The release member cap and needle assembly may be configured to cooperate with the same guide rib or guide rail of the aperture. For example, the cap and the needle assembly may both comprise a guide rib, and the aperture may comprise a guide rail configured to cooperate with both the guide rib of the cap and the guide rib of the needle assembly.

The release member may be movable relative to the housing between a primed position in which the retaining arms are arranged to cooperate with the housing to retain the needle assembly in a primed position, and a deployed position in which the retaining arms are arranged to release the needle assembly to deploy the needle.

According to a second aspect of the invention, there is provided a single use lancing device. The lancing device comprises: a housing defining a cavity; a needle assembly comprising a needle and a needle holder, the needle assembly located within the cavity; a release member; and a spring arranged to bias the needle assembly in a forward direction relative to the release member. The release member comprises retaining arms and is movable relative to the housing between a primed position in which the retaining arms are arranged to cooperate with the housing to retain the needle assembly in a primed position and a deployed position in which the retaining arms are arranged to release the needle assembly to deploy the needle. The release member further comprises a primary latching projection arranged to prevent the release member being moved in a rearward direction when the release member is in the primed position so that the release member cannot be removed from the housing.

The lancing device may further comprise a secondary latching projection configured to allow the release member to move into the deployed position from the primed position. The secondary latching projection may be positioned on one of the retaining arms of the release member. Preferably a pair of secondary latching projections is provided, which may be positioned on opposing retaining arms relative to each other. A secondary latching projection may be provided on each retaining arm of the release member.

The secondary latching projection may be arranged to prevent the movement of the release member from the deployed position back to the primed position, and optionally prevent disassembly of the release member from the housing. Both the primary and secondary latching projections may work in combination to prevent the movement of the release member from the deployed position back to the primed position and/or prevent disassembly of the release member from the housing.

It may be preferable or advantageous for the lancing device to comprise the primary latching projection and the secondary latching projection.

Providing both primary and secondary latching projections may allow for each to be better optimised for their respective function compared to a device comprising just one projection performing both functions. Namely, the primary latching projection prevents the release member being removed from the housing, and the secondary latching projection may prevent the release member being returned from the deployed position to the primed position.

The primary latching projection may remain in the first receiving portion of the housing in the primed and deployed position. Thus, the primary latching projection may only need to slide past a lip of the housing at the rear opening (and not past body portions that separate the receiving portions). Therefore, the primary latching projection may be made larger or more resilient compared to a device with only one projection. This may result in the device being harder to disassemble.

The secondary latching projection may not be required to prevent disassembly (as the primary latching projection is provided for this). Thus, the secondary latching projection may only cooperate with the housing when in the deployed position and may prevent the release member from returning from the deployed to the primed position. Alternatively, the reliance on the secondary latching projection to function to prevent disassembly may be reduced. Therefore, the secondary latching projection may be made smaller, less resilient, more flexible or more rounded than compared to a device with only one projection (i.e. only the primary latching projection). This may mean that activation of the device (movement from the primed to the deployed position) may be easier, and the retaining arms may be better able to flex when released.

In an embodiment wherein the secondary latching projection does not need to prevent disassembly (i.e., because the primary latching projection provides this function), the “secondary latching projection” may otherwise be termed a “secondary projection”, i.e., because it may not require a latching function.

The primary and/or secondary latching projections may be provided as a pair. E.g., a pair of primary latching projections and/or a pair of secondary latching projections. Therefore, in one embodiment, there may be at least four latching projections that may prevent disassembly of the release member from the housing and/or prevent return from the deployed position to the primed position. In another embodiment, there may be at least two latching projections (i.e., two primary latching projections) that may prevent disassembly of the release member from the housing and/or prevent return from the deployed position to the primed position, and at least two secondary projections, which are arranged to control the retaining arms during deployment of the needle assembly from the retaining member.

The primary latching projections in a pair may be positioned on opposing surfaces of the release member. Additionally or alternatively, the secondary latching projections in a pair may be positioned on opposing surfaces of the release member, such as on opposing retaining arms.

The housing may comprise: a first receiving portion configured to receive the primary latching projection when the release member is in the primed position; a second receiving portion configured to receive the secondary latching projection when the release member is in the primed position; and a third receiving portion configured to receive the secondary latching projection when the release member is in the deployed position.

The first, second and third receiving portions may be apertures or windows through the body of the housing. Alternatively, the receiving portions may be indents or recesses on an inner surface of the housing body.

The primary latching projection may be received by the first receiving portion when the release member is in the deployed position. Alternatively, the primary latching projection may be received by the second or third receiving portions when the release member is in the deployed position. The primary latching projection may be arranged to prevent the movement of the release member from the deployed position back to the primed position.

In an embodiment comprising pairs of primary and/or secondary latching projections, the housing may comprise corresponding receiving portions for each of the respective latching projections.

The lancing device may further comprise a removable safety member configured to prevent the release member being moved from the primed position to the deployed position.

The removable safety member may comprise a collar configured to fit circumferentially around a portion of the release member, wherein a primary end of the collar is connected to a second end of the collar via a weakened shear portion. The collar may be configured to fit around the body of the release member proximate to a rear end.

The weakened shear portion may comprise an overlapping region between the first end of the collar and the second end of the collar. The weakened shear portion may comprise at least one projection, tab or conical shaped protrusion connecting the first end and second end of the collar at the overlapping region.

The first and second end may overlap radially such that one end is positioned outside of the other end relative to the lancing device, when the collar is fitted to the release member. Alternatively, the first and second end may overlap longitudinally such that one end is positioned forward of the other end relative to lancing device, when the collar is fitted to the release member.

The needle assemble may further comprise a needle cover which is formed integrally with the needle holder with a shear portion therebetween so that the needle cover can be removed from the needle and needle holder.

When the release member is in the primed position, part of the cover may protrude through a hole in the front end of the housing.

The hole may have a diameter sufficient to allow passage of the needle assembly. The hole may have a diameter of least 2.5 mm, or at least 3 mm, or at least 3.2 mm. The hole may preferably have a diameter of 3.2 mm. The diameter of the hole may be within a range of ±5%, or ±10%, or ±20% of a preferred diameter.

Rotating the cover relative to the needle holder may break the shear portion and allow the cover to be removed from the needle.

The spring may be maintained in a compressed state when the needle assembly is in the primed position. The spring may have a relaxed or natural state in which the needle is retained completely within the housing.

The spring may be arranged so that on deployment of the needle the spring moves to an extended state in which the needle projects from the front end of the housing. The spring may subsequently return to the relaxed state to retract the needle into the housing.

The housing may further comprise a retaining surface arranged to retain the retaining arms in a retaining position when the release member is in its primed position. The housing may have openings into which the retaining arms can move to release the needle assembly when the release member is in the deployed position. The openings may be apertures windows through the housing body or indents/recesses on an inner surface of the housing body.

The release member may comprise at least one guide rib and the housing may comprise at least one corresponding guide rail, such that the guide rib slidably moves along the guide rail when the release member moves relative to the housing. Alternatively, the release member may comprise a guide rail and the housing may comprise a guide rib.

Features of the first aspect may be applied to the device of the second aspect, and vice versa. For example, the lancing device of the first aspect may further comprise a primary latching projection arranged to prevent the release member being moved in a rearward direction when the release member is in the primed position so that the release member cannot be removed from the housing.

### DETAILED DESCRIPTION

Embodiments of the invention will be described, purely by way of example, with reference to the accompanying drawings, in which:

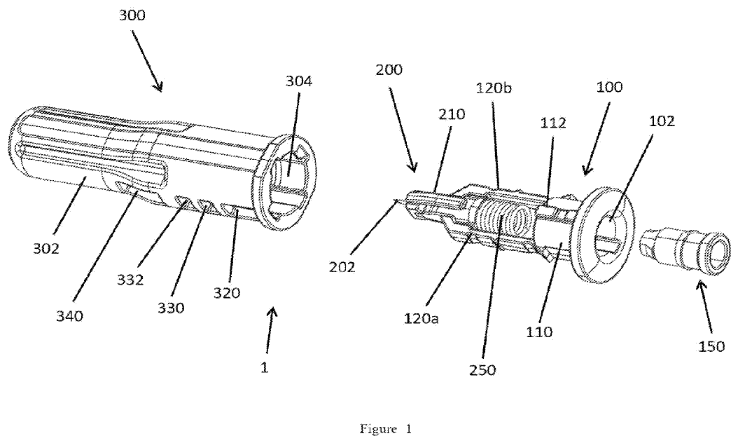

FIG. 1 shows an exploded view of a lancing device according to an embodiment of the present invention;

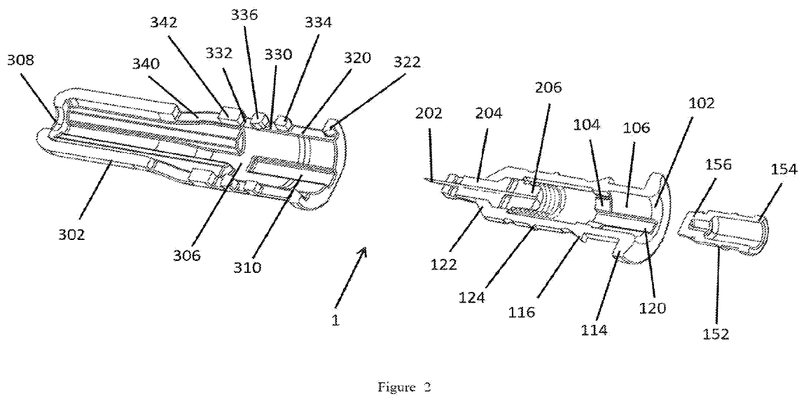

FIG. 2 shows a perspective view of the lancing device of FIG. 1;

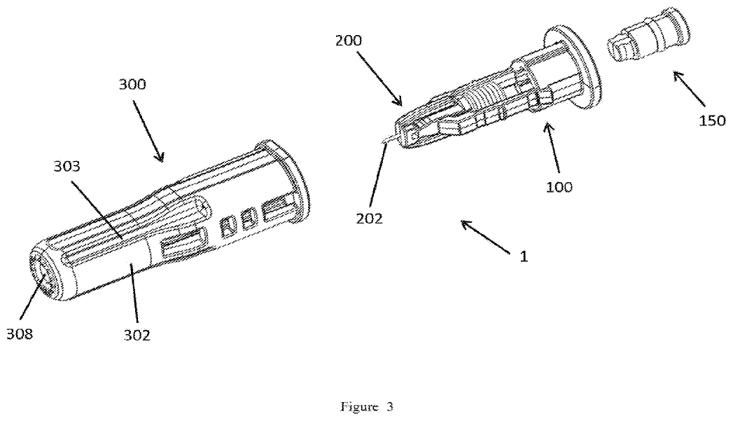

FIG. 3 shows a further view of the lancing device of FIG. 1;

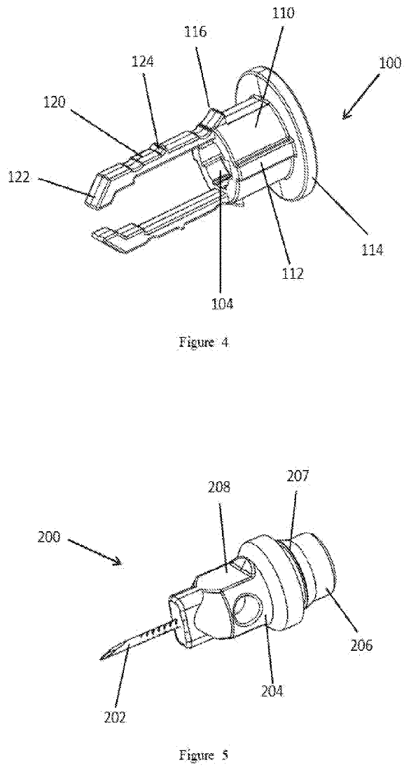

FIG. 4 shows a release member forming part the lancing device of FIG. 1;

FIG. 5 shows a needle assembly forming part of the lancing device of FIG. 1;

FIG. 6 shows a perspective view of the lancing device of FIG. 1 in a primed position;

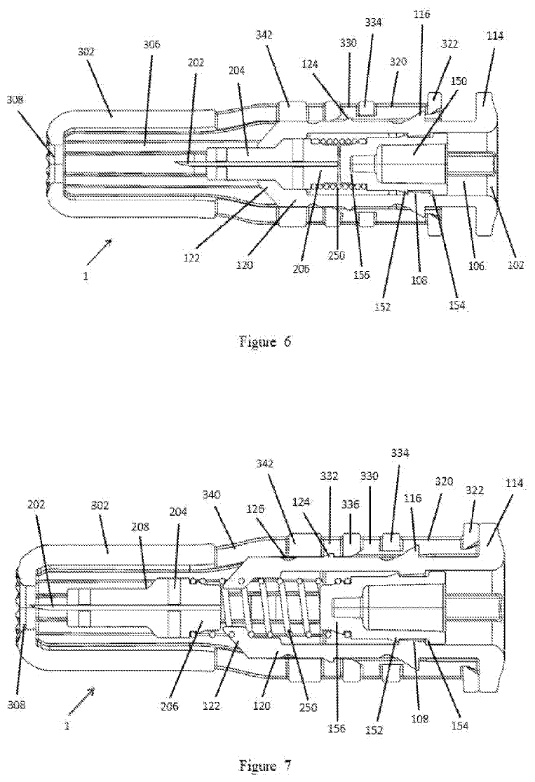

FIG. 7 shows a perspective view of the lancing device of FIG. 1 in a deployed position;

FIG. 8 shows a needle cover attached to the needle assembly of FIG. 5;

FIG. 9 shows a perspective view of the needle cover of FIG. 8;

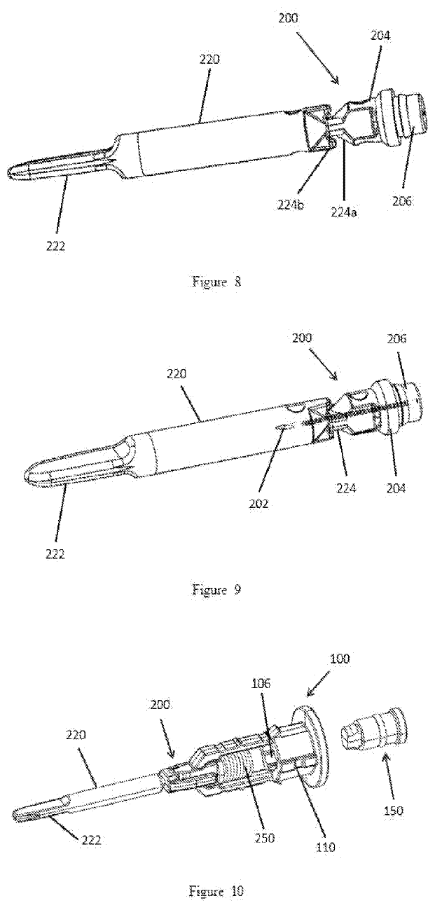

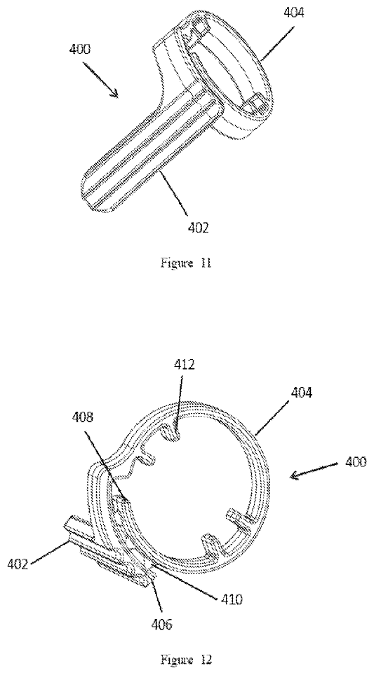

FIG. 10 shows part of a lancing device with a needle covering according to an embodiment of the present invention;

FIG. 11 shows a removable safety member;

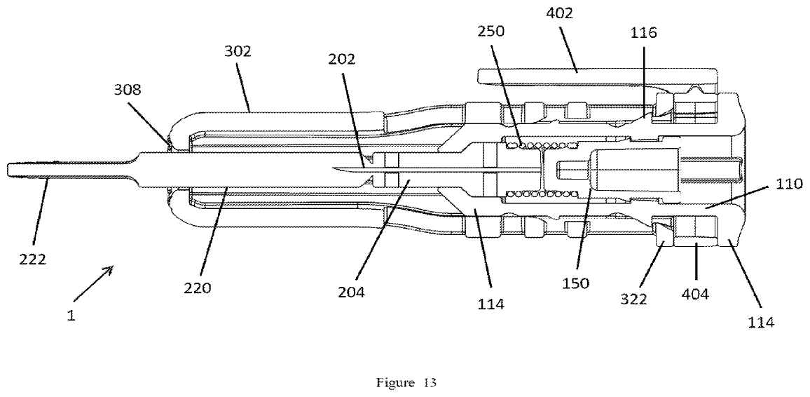

FIG. 12 shows a further view of the removable safety member of FIG. 11;

FIG. 13 shows the lancing device in the primed position as shown in FIG. 6 with a needle cover and removable safety member attached.

Referring to FIGS. 1, 2 and 3, a lancing device 1 is shown in a semi-deconstructed state. The lancing device comprises a release member 100, the release member comprising a body portion 110 and retaining arms 120a and 120b. The body portion 110 has a rear opening 102 and a forward opening 104. The body portion 110 may be substantially hollow and cylindrical, having an aperture 106 that is accessible at either end of the release member body via the rear opening 102 and forward opening 104.

The lancing device 1 further comprises a release member cap 150. The release member cap 150 is configured to be received by the rear opening 102 of the release member body 110, so that the cap 150 fits at least partially within the aperture 106 within the release member body 110.

The lancing device further comprises a needle assembly 200. The needle assembly 200 comprises a needle 202 supported in a needle holder 204. The needle holder 204 may be made of plastic material and moulded over the base of the needle 202. The needle assembly 200 is configured to be positioned at least partially between the retaining arms 120. The retaining arms 120 may comprise a tapered end portion 122 configured to engage with a portion of the needle holder 204. The needle 202 may be sterilized, for example using gamma radiation, before use.

The lancing device 1 further comprises a housing 300. The housing 300 comprises a housing body 302 that may be substantially hollow and cylindrical, having a cavity 306. The cavity 300 is connected to a hole 308 in a front end of the housing body 302 and has rear opening 304 at a rear end of the housing body 302. The rear opening 302 may be large relative to the diameter of the housing body 302, such that the housing 300 is substantially open at the rear end. The hole 308 may be smaller than the rear opening 304. The hole 308 may be configured such that a user cannot easily gain access to the cavity 306 via the hole 308 (e.g., small enough so that a person cannot insert a finger through the hole 308). The front end of the housing 300 and the hole 308 may be configured for contact with a patient's skin. The housing body 302 may have ribs, deformations, or shaped portions 303. The shaped portions 303 may strengthen the housing 300 or may provide grip surfaces for the user.

The lancing device 1 further comprises a spring 250. The spring 250 is used to deploy the needle assembly 200 during use, as discussed in detail later. The needle holder 204 may comprise a spring mounting 206. A first end of the spring 250 may fit over the needle holder spring mounting 206, as shown in FIG. 2. The release member cap 150 may also comprise a spring mounting 156. The spring 250 may be retained by the two spring mountings such that when in an assembled configuration, the spring is prevented from moving laterally. The spring 250 may be a helical spring as shown, or any other type of spring.

FIGS. 1, 2 and 3 show the lancing device 1 in a semi-assembled state. The release member 100 and needle assembly 200 are separate components and are assembled to form the arrangement shown. The rear opening 102, forward opening 104 and aperture 106 of the release member body 110 may be configured such that the needle assembly 200 and spring 250 may be passed through the aperture 106 from the rear direction. To position the needle assembly 200 within the retaining arms 120 as shown, the needle assembly 200 may be pushed through the aperture 106 from the rear direction. The needle assembly 200 may then be advanced further in the forward direction, such that a forward part of the needle holder 204 is advanced beyond tapered portion 122 of the retaining arms 120. The needle assembly 200 may be advanced to a position such that a rear portion of the needle holder 204 with a larger diameter abuts the tapered portion 122 of the retaining arms 120, the tapered portion 122 preventing the needle assembly 200 being advanced further. The retaining arms 120 may be flexible and resilient.

The spring 250 may also be passed through the aperture 106 from the rear direction such that it is positioned between the retaining arms 120 as shown. The spring 250 may be attached the spring mount 206 of the needle assembly 200 prior to being passed through the aperture 106 (such that the needle assembly 200 and spring 250 are positioned simultaneously). Alternatively, the needle assembly 200 may be positioned first, and subsequently the spring 250 passed through the aperture 106 such that it fits over the needle mounting 206.

Following insertion of the needle assembly 200 and spring 250, the release member cap 150 may be inserted into the aperture 106 from a rearward direction. When the release member cap 150 is inserted into the release member 100, a second end of the spring 250 may fit over the cap spring mounting 156.

The release member 100, release member cap 150, needle assembly 200 and spring 250 may be inserted into the housing 300 via the rear opening 304 of the housing body 302. The release member 100, release member cap 150, needle assembly 200 and spring 250 may be assembled as described above prior to insertion into the housing 300. Alternatively, the release member 100 is first inserted into the housing 300. Subsequently, the release member cap 150, needle assembly 200 and spring 250 may be inserted into the release member 100/housing 300 via the rear opening 102 of the release member 100 as described above.

The release member body 110 may comprise a guide rib 112 and the housing 300 may comprise a guide rail 310 within the cavity 306. The guide rib 112 may be a projection that runs at least partially along an outer surface of the release member body 110. The guide rail 310 may be an indentation in an inner surface of the housing body 302 that runs at least partially along the length of the cavity 306. The guide rib 112 and guide rail 310 may be configured such that the guide rib 112 is slidably received within the guide rail 310 when the release member 100 is received within the housing 300.

The needle assembly 200 may comprise a guide rib 210 and the release member body 110 may comprise a guide rail 120 within the aperture 106. The guide rib 210 may be a projection that runs at least partially along an outer surface of the needle holder 204. The guide rail 120 may be an indentation in an inner surface of the release member body 110 that runs at least partially along the length of the aperture 106. The guide rib 210 and guide rail 120 may be configured such that the guide rib 210 is slidably received within the guide rail 120 when the needle assembly 200 is received within the release member 100.

The release member cap 150 may comprise a guide rib. The guide rib may be a projection that runs at least partially along an outer surface of the release member cap 150. The guide rib may be configured such that the guide rib is slidably received within the guide rail 120 when the release member cap 150 is inserted into release member 100. The guide rail of the release member cap 150 may be configured to be received by the same guide rail 120 as the needle assemble 200. Alternatively, the release member body 110 may comprise an additional guide rail for the release member cap 150 guide rib.

Each guide rib may comprise a pair of guide ribs arranged on opposing sides of the respective bodies/components. Each guide rail may correspondingly comprise a pair of guide rails arranged on opposing sides of the release member body 110 or housing 300. The guide ribs and rails may assist in the assembly of the various components. The guide ribs and rails may ensure that the components are correctly aligned during assembly. The guide ribs and rails may prevent rotation of the components once assembled.

The release member cap 150 may comprise a latching mechanism 152 and a stopping mechanism 154 to assist in positioning the release member cap 150 within the release member body 110.

The release member 100 may comprise a neck portion 114, a primary latching projection 116 and a secondary latching projection 124. The housing 300 may comprise a first receiving portion 320, second receiving portion 330, third receiving portion 332 and release aperture 340. These receiving portions/apertures may be defined by body portions 322, 334, 336 and 342. The latching projections may be configured to engage with the receiving portions/apertures when the release member 100 is received by the housing 300. The function of the various latching projections and the corresponding receiving portions/apertures will be described in further detail later.

Referring to FIG. 4, an example of a release member 100 is shown. The release member comprises a body 110. The body 110 has a forward opening 104 that leads to an aperture within the release member body 110. As discussed previously, the aperture extends through the release body portion along a central longitudinal axis of the release member from a rear opening to the forward opening 104. The release member body 110 may comprise a flared neck portion 114 which has a larger diameter than the body 110. The release member body 110 may further comprise a guide rib 112 on an outer surface of the body 110. The guide rib 112 may run along the length of the body 110 from the neck portion 114 to a forward end of the body 110.

The release member 100 may comprise a pair of retaining arms 120 having a tapered end portion 122. The retaining arms 120 may extend forward from the body 110.

The release member 100 may have a primary latching projection 116. The primary latching projection 116 may be located where the retaining arm 120 is coupled to the body 110. The release member 100 may further comprise a secondary latching projection 124. The secondary latching projection 124 may protrude from an outer surface of the retaining arm 120.

Referring to FIG. 5, an example of a needle assembly 200 is shown. The needle assembly 200 comprises a needle 202 coupled to a needle holder 204. The needle holder 204 may comprise retaining surfaces 208. The retaining surfaces 208 may comprise a flat portion and an angled portion configured to correspond to the tapered portion 122 of the retaining arms 120. When the needle assembly 200 is positioned within the release member 100 (as shown in FIG. 1), the tapered retaining arms 120 may engage with the retaining surfaces 208 such that the needle assembly 200 cannot be further advanced in a forward direction. The angled portion of the receiving surface 208 may abut the corresponding tapered portion 122 of the retaining arm 120.

The body of the needle holder 204 may comprise a spring mount 206 as discussed previously. The spring mount 206 may comprise a protrusion or flange 207. A rear side of the flange 207 may be tapered such that the spring 250 can be pushed over the flange from the rear direction. A forward side of the flange 207 may be at 90° to the spring mount 206 such that once the spring 250 is pushed over the flange 207 the spring 250 cannot easily be removed. The spring mount 206 may thus retain an end of the spring 250 in a longitudinal position relative to the needle assembly 200. The spring mount 156 of the release member cap 150 (as shown in FIG. 2), may have a similar flange for retaining an opposing end of the spring 250 in a longitudinal position relative to the release member 100.

Referring to FIG. 6, a lancing device 1 is shown in a primed position. The lancing device 1 may comprise features as described in earlier Figures.

In the primed position, the needle 202 is contained within the cavity 306 of the housing 300. The needle holder 204 is retained between the retaining arms 120. The spring 250 is in compression.

The release member cap 150 may be retained in a position partially within the release member body 110. The release member body 110 may comprise a retaining protrusion 108. The protrusion 108 may be a retaining ring that protrudes into the aperture 106 around the circumference at the forward opening 104. The release member cap may comprise a latching mechanism 152. The latching mechanism 152 may have a tapered front face such that the latching mechanism can be advanced past the retaining ring 108 from a rear direction. The latching mechanism 152 may have a rear face that is at 90° to the cap 150, such that once the latching mechanism is advanced past the retaining ring 108, the release member cap 150 cannot be pulled back into the aperture 106.

The release member cap 150 may further comprise a stopping mechanism 154. The stopping mechanism 154 may comprise a protruding ring near the rear end of the release member cap. The stopping mechanism 154 may prevent the release member cap 150 being advanced relative to the release member 100 beyond a position (i.e., the protruding ring may not be advanced past the retaining ring 108). The latching mechanism 152 and stopping mechanism 154 may act cooperatively to retain the release member cap 150 in a desired position once the cap 150 has been sufficiently advanced through the aperture 106 from the rear direction.

In the latched position, the retaining arms 120 may engage the needle holder 204. The tapered portions 122 may engage the retaining surfaces 208. In the primed position, the retaining arms 120 may be retained by body portions 342 of the housing 300. The body portions 342 may prevent the retaining arms 120 from being deformed outwardly, thereby preventing the needle holder 204 from advancing forward past the tapered portions 122.

In this configuration, with the release member cap 150 retained in a desired position within the release member body 110 and the needle assembly 200 prevented from moving forward by the retaining arms, the spring 250 is retained in compression. The spring 250 may be prevented from moving laterally by the spring mounts 156, 206.

The release member 100 may comprise primary latching projection 116. The primary latching projection 116 may be received in the first receiving portion 320 when the lancing device 1 is in the primed position. The primary latching projection 116 may have a tapered front face and the housing opening 304 and/or the body portion 322 may have a tapered inner surface such that the release member 100 can be inserted into the housing 300. The primary latching projection 116 may have a rear face that is at 90° to the release member body 110, such that when the release member 100 is inserted into the housing 300 it cannot be pulled back out via the opening 304. The primary latching projection 116 may thus prevent disassembly of the lancing device 1 once arranged in the primed position.

The release member 100 may further comprise a secondary latching projection 124. The secondary latching projection 124 may be received in the second receiving portion 320 when the lancing device is in the primed position. The secondary latching projection 124 may have a tapered front face such that the release member 100 can be slidably advanced within the housing 300. The central part of the retaining arms 120 on which the secondary latching projection 124 is formed may flex inwards to allow the secondary latching projection 124 to slide forward within the housing. The primary latching projection 124 may have a rear face that is at 90° to the retaining arm 120, which may engage against body portions to prevent rearward sliding motion of the release member 100.

The secondary latching projection 124 may not engage with the body portion 334, as shown in FIG. 6. Alternatively, the secondary latching projection 124 may engage with the body portion 334 such that the secondary latching projection 124 abuts the body portion 334 to prevent rearward sliding motion of the release member 100.

Referring to FIG. 7, the lancing device 1 is shown in a deployed or fired position. The lancing device 1 may comprise features as described in earlier Figures.

The lancing device 1 may be moved from the primed position to the deployed position by advancing the release member 100 relative to the housing 300. This may be achieved by a user pushing on the rear end of the release member 100.

When the release member 100 is advanced from the primed position to the deployed position, a forward portion of the retaining arms 120 is advanced past the body portion 342. The release aperture 340 may allow the retaining arms 120 to deform outward so that widest part of the needle holder 204 can pass between the tapered end 122 of the retaining arms 120. The force of the compressed spring 250 is sufficient to push the needle holder 204 forwards relative to release member 100. The angled portion of the needle holder engaging surface 208 may assist in prising apart the retaining arms 120. The retaining arms 120 may have indents 126 that may assist with the outward deformation of the retaining arms 120.

The force of the spring 250 is sufficient to push the needle holder 204 past the tapered portions 122 of the deformed retaining arms 120 and accelerate needle assembly 200 until the spring 250 reaches its natural or relaxed length (as shown in FIG. 7). The momentum of the needle holder 204 is sufficient to carry the needle 202 beyond this natural length so that the end of the needle 202 projects through the hole 308 at the front of the housing 300. The needle holder 204 and hole 308 may be configured so that the needle holder 204 contacts the housing body 302 to prevent the needle holder 204 leaving the cavity 306.

With the needle 202 projecting from the hole 308, the spring 250 is fully extended. Even when extended fully, the spring 250 may be retained on the spring mounts 156, 206 so that the spring does not come loose.

The extended spring 250 then returns to its natural length, retracting the needle 202 to the deployed position shown in FIG. 7. In the deployed position, the needle 202 is contained within the cavity 306 of the housing 300. This prevents the needle 202 from coming into contact with another person after use.

The release member 100 may be prevented from being further advanced beyond the deployed position by the neck portion 114 abutting the housing body portion/opening lip 322.

In the deployed position, the secondary latching projection 124 may be received by the third receiving portion 332. The rear face of the secondary latching projection 124 may abut the body portion 336 to prevent the release member 100 being slid backwards from the deployed position to the primed position. This prevents the release member 100 from being pulled backwards, and therefore prevents re-use of the device.

The combination of the neck 114 and secondary latching projection 124 may prevent any substantial movement of the release member 100 relative to the housing either forwards or backwards, essentially locking the now used lancing device 1. The lancing device 1 may then be discarded.

In the deployed position, the primary latching projection 116 may be received by the first receiving portion 320, as shown in FIG. 6. Alternatively, the primary latching projection 116 may be received by the second receiving portion 330, wherein the body portion 334 further prevents the release member 100 being pulled backwards.

Referring to FIGS. 8 and 9, a needle assembly 200 is shown further comprising a needle cover 220. The needle cover 220 may be of the same material as the needle holder 204 and may be formed my over-moulding onto the needle 202 at the same time as the needle holder 204. Indeed, the needle holder 204 and cover 220 may be formed integrally with each other, with a shear portion 224 formed between them which can be broken to separate them and remove the cover 220 from the needle 202.

The needle cover 220 may have a cylindrical body and project forward from the front end of the needle 202. The front end of the needle cover body may be flattened to form a grip portion 222 that can be easily held by a user such that the user can apply a twisting torque to the needle cover 220. Cam surfaces 224a on the needle holder 204 and cam projections 224b on the cover 220 may be arranged to cooperate on rotation of the cover 220 relative to the needle holder 204 to urge the cover 220 forwards away from the needle holder 204, thereby aiding in the separation of the cover 220 from the needle holder 204.

Referring to FIG. 10, an assembly is shown comprising a release member 100, release member cap 150 and needle assembly 200. The release member 100, release member cap 150 and needle assembly 200 may comprise any of the features described in previous examples (as shown in FIGS. 1 and 2, for example). The needle assembly 200 comprises a needle cover 220. The cover 220 and handle 222 are configured so that they can be passed through the aperture 106 of the release member body 110. The needle assembly 200 (including cover 220), spring 250 and release member cap 150 may therefore be inserted into the release member 100 for assembly as discussed previously.

Referring to FIGS. 11 and 12, an example of a removable safety member 400 is shown. The safety member 400 comprises a handle or tab portion 402 and a collar portion 404. The collar portion 404 may be configured to fit circumferentially around a portion of the release member 100, for example around the release member body 110 proximate to the neck 114 at the rear end.

The collar portion 404 may comprise a first end portion 406 and a second end portion 408. The first end 406 and second end 408 may overlap and may be coupled to one another by a weakened tearable shear portion 410. The shear portion 410 may comprise a plurality of ribs, protrusions or cones, each with a thickness less than that of the main collar 404. The shear portion 410 may be broken by the user pulling on the handle 402 while the collar is fitted around the release member body 110. With the shear portion 410 broken, the first end 406 and the second end 408 are no longer attached and the collar 404 may be removed from the release member 100.

The collar 404 may comprise one or more ribs 412 on the inner circumference of the collar 404. The ribs 412 may mean that less material is needed to form the collar 404 compared to a solid collar with the same thickness at the ribbed collar 404. The ribs 412 may also allow the collar 404 to flex more than a solid collar that contacts the release member body 110 around the entire circumference. This additional flexing may allow for easier tearing of the shear portion 410.

Referring to FIG. 13, an example of a lancing device 1 is shown. The lancing device in FIG. 13 may comprise any of the features presented in previous examples (as shown in FIGS. 6 and 7, for example). The lancing device 1 is provided with a needle cover 220 and a removable safety member 400 such that the lancing device 1 is in a safety or storage configuration. The release member 100 is in the primed position.

The collar 404 of the safety member 400 is circumferentially fitted around the body 110 of the release member 100. The collar 404 may be fitted at a location near the neck 114 of the release member 100. The collar 404 may prevent the release member 100 being advanced through the housing body 302 by being positioned between the neck 114 and the housing body portion/lip 322, thus keeping the neck 114 and housing body 302 separated at a fixed distance.

The user may activate the lancing device 1 by twisting the needle cover handle 222 so that the cover 220 separates from the needle holder 204. The user may then remove the cover 220 from the housing body 302 via the hole 308. The user may then pull on the safety member tab 402 to remove the collar 404 from the release member body 110. Following these two steps, the lancing device shall be configured in the primed position and ready for use as shown in FIG. 6.

While in the storage configuration, the lancing device 1 is inactive. The removable safety member 400 prevents the release member 100 being advanced from the primed position to the deployed position. The primary latching protrusion 116 of the release member 100 and the latching mechanism 152 of the release member cap 150 prevent disassembly of the device. The housing body 302 and cover 220 conceal the needle 202 which may prevent damage, contamination and accidental user contact of the needle 202.

The examples presented show devices which have substantial two-fold symmetry through a plane running along the longitudinal axis of the device (e.g., the release member 100 has two opposing retaining arms 120, the housing 300 has opposing receiving portions/apertures 320, 330, 332, 340, the various guide rails and ribs are shown in opposing pairs, etc.). However, the device may comprise any number of these features and these features may not be arranged symmetrically. The same number of each feature may not be required (so long as cooperating features may still cooperate). For example, the device may comprise three retaining arms and three sets of housing receiving portions, but only one release member guide rib and housing aperture guide rail.

Although example embodiments have been described, these are not intended to limit the scope of the invention, which should be determined with reference to the accompanying claims.

1. A single use lancing device comprising:
a housing defining a cavity;
a needle assembly comprising a needle and a needle holder, the needle assembly located within the cavity;
a release member comprising:
a body portion, the body portion having an aperture passing through the body portion along a central longitudinal axis of the release member from a rear opening to a forward opening; and
retaining arms;
a release member cap configured to be received by the rear opening such that the release member cap at least partially fits within the aperture; and
a spring arranged to bias the needle assembly in a forward direction relative to the release member.

2. The lancing device of claim 1, wherein the release member cap comprises a latching mechanism configured to engage with the release member such that when the release member cap is received by the rear opening, removal of the release member cap via the rear opening is prevented.

3. The lancing device of claim 2, wherein the release member and/or release member cap comprises a stopping mechanism configured to prevent the release member cap being advanced through the aperture beyond a desired position.

4. The lancing device of claim 1, wherein the release member cap comprises at least one guide rib and the aperture of the release member comprises at least one corresponding guide rail, such that the guide rib slidably moves along the guide rail when the release member cap is received by the aperture.

5. The lancing device of claim 1, wherein the release member cap is configured to seal the aperture.

6. The lancing device of claim 1, wherein the aperture of the release member is configured to allow the needle assembly and spring to be passed through the aperture from the rear opening to the front opening.

7. The lancing device of claim 6, wherein the needle assembly comprises at least one guide rib and the aperture of the release member comprises at least one corresponding guide rail, such that the needle assembly guide rib slidably moves along the guide rail when the needle assembly is passed through the aperture.

8. The lancing device of claim 1, wherein the release member is movable relative to the housing between a primed position in which the retaining arms are arranged to cooperate with the housing to retain the needle assembly in a primed position, and a deployed position in which the retaining arms are arranged to release the needle assembly to deploy the needle.

9. A single use lancing device comprising:
a housing defining a cavity;
a needle assembly comprising a needle and a needle holder, the needle assembly located within the cavity;
a release member; and
a spring arranged to bias the needle assembly in a forward direction relative to the release member;
wherein the release member comprises retaining arms and is movable relative to the housing between a primed position in which the retaining arms are arranged to cooperate with the housing to retain the needle assembly in a primed position and a deployed position in which the retaining arms are arranged to release the needle assembly to deploy the needle; and
further comprising a primary latching projection arranged to prevent the release member being moved in a rearward direction when the release member is in the primed position so that the release member cannot be removed from the housing.

10. The lancing device of claim 9, further comprising a secondary latching projection configured to allow the release member to move into the deployed position from the primed position.

11. The lancing device of claim 10, wherein the secondary latching projection is arranged to prevent the movement of the release member from the deployed position back to the primed position.

12. The lancing device of claim 10, wherein the housing comprises: a first receiving portion configured to receive the primary latching projection when the release member is in the primed position; a second receiving portion configured to receive the secondary latching projection when the release member is in the primed position; and a third receiving portion configured to receive the secondary latching projection when the release member is in the deployed position.

13. The lancing device of claim 9, further comprising a removable safety member configured to prevent the release member being moved from the primed position to the deployed position.

14. The lancing device of claim 13, wherein the removable safety member comprises a collar configured to fit circumferentially around a portion of the release member, wherein a first end of the collar is connected to a second end of the collar via a weakened shear portion.

15. The lancing device of claim 14, wherein the weakened shear portion comprises an overlapping region between the first end of the collar and the second end of the collar.

16. The lancing device of claim 9, further comprising a needle cover which is formed integrally with the needle holder with a shear portion therebetween so that the needle cover can be removed from the needle and needle holder.

17. The lancing device of claim 16, wherein when the release member is in the primed position, part of the cover protrudes through a hole in the front end of the housing.

18. The lancing device of claim 16, wherein rotating the cover relative to the needle holder breaks the shear portion and allows the cover to be removed.

19. The lancing device of claim 9, wherein the spring is maintained in a compressed state when the needle assembly is in the primed position and has a relaxed state in which the needle is retained completely within the housing.

20. The lancing device of claim 19 wherein the spring is arranged on deployment of the needle to move to an extended state in which the needle projects from the front end of the housing, before returning to the relaxed state to retract the needle into the housing.

21. The lancing device of claim 9, wherein the housing:
comprises a retaining surface arranged to retain the retaining arms in a retaining position when the release member is in its primed position; and
defines openings into which the retaining arms can move to release the needle assembly when the release member is in the deployed position.

22. The lancing device of claim 9, wherein the release member comprises at least one guide rib and the housing comprises at least one corresponding guide rail, such that the guide rib slidably moves along the guide rail when the release member moves relative to the housing.
